(19) United States
(12) Patent Application Publication
(10) Pub. No.: US 2026/0158906 A1
(43) Pub. Date: Jun. 11, 2026

(54) METHOD OF PREVENTING DRUNK DRIVING AND APPARATUS FOR PERFORMING THE METHOD

(71) Applicant: Electronics and Telecommunications Research Institute, Daejeon (KR)

(72) Inventors: Hyowoong NOH, Daejeon (KR); Dae-Sik LEE, Daejeon (KR); Junyeong LEE, Daejeon (KR); Byeongju LEE, Daejeon (KR)

(21) Appl. No.: 19/344,342

(22) Filed: Sep. 29, 2025

(30) Foreign Application Priority Data
Dec. 5, 2024 (KR) 10-2024-0179775
Mar. 27, 2025 (KR) 10-2025-0039763

(51) Int. Cl. B60K 28/06; A61B 5/00; A61B 5/08; B60K 28/04

(52) U.S. Cl. CPC B60K 28/06; A61B 5/082; A61B 5/4845; A61B 5/6893; B60K 28/04; A61B 2503/22

(57) ABSTRACT

Provided is a method of preventing drunk driving of a driver who rides in a vehicle. The method includes sensing actions of the driver, determining whether a sobriety test of the driver is possible based on a sensed result, obtaining information on whether the driver is intoxicated when it is determined that the sobriety test of the driver is possible, and controlling the vehicle based on the information, in which the sensing of the actions includes sensing whether the driver sits in a driver's seat of the vehicle, sensing whether the driver closes all doors of the vehicle, sensing whether the driver fastens a seat belt in the driver's seat of the vehicle, sensing whether the driver steps on a brake of the vehicle, and sensing whether the driver grips a steering wheel of the vehicle.

### CROSS-REFERENCE TO RELATED APPLICATIONS

This application claims the benefit of Korean Patent Application No. 10-2024-0179775, filed on Dec. 5, 2024, and Korean Patent Application No. 10-2025-0039763, filed on Mar. 27, 2025, in the Korean Intellectual Property Office, the entire disclosures of which are incorporated herein by reference for all purposes.

### BACKGROUND

### 1. Field of the Invention

One or more embodiments relate to a method of preventing drunk driving and an apparatus for performing the method.

### 2. Description of the Related Art

A portable breathalyzer may measure alcohol in exhaled breath using an electrochemical gas sensor. The principle is that an alcohol molecule in exhaled breath is oxidized by a platinum anode electrode and current flows, and strong current may flow as an alcohol molecule in exhaled breath increases. A portable breathalyzer may calculate blood alcohol concentration by measuring the strength of current. A portable breathalyzer used by the police is inspected every four months by the Korea Road Traffic Authority in accordance with the National Police Agency regulations, and an apparatus with a large error undergoes two-stage calibration, but there may be many limitations in the current sobriety test method.

The currently implemented sobriety test method may be a method of measuring whether a driver is intoxicated by stopping all vehicles passing through a specific section. This method may have the disadvantage of mobilizing a large number of people on the road for a sobriety test. Accordingly, there may be a disadvantage that additional traffic accidents may occur due to a sobriety test. This sobriety test method may instill in a driver the perception that the driver is not caught in the crackdown on drunk driving as long as the driver avoids a specific section and time zone in which a sobriety test is performed. Accordingly, the effect of the crackdown on drunk driving may be reduced and drunk driving may not be prevented.

The above description is information the inventor(s) acquired during the course of conceiving the present disclosure, or already possessed at the time, and is not necessarily art publicly known before the present application was filed.

### SUMMARY

Embodiments may sense the actions of a driver who rides in a vehicle.

Embodiments may determine whether a sobriety test of a driver is possible based on the sensed actions of the driver.

Embodiments may start a sobriety test and obtain information on whether a driver is intoxicated when the sobriety test of the driver is possible.

However, the technical goals are not limited to those described above, and other technical goals may be present.

According to an aspect, there is provided a method of preventing drunk driving of a driver who rides in a vehicle, the method including sensing actions of the driver, determining whether a sobriety test of the driver is possible based on a sensed result, obtaining information on whether the driver is intoxicated when it is determined that the sobriety test of the driver is possible, and controlling the vehicle based on the information, in which the sensing of the actions includes sensing whether the driver sits in a driver's seat of the vehicle, sensing whether the driver closes all doors of the vehicle, sensing whether the driver fastens a seat belt in the driver's seat of the vehicle, sensing whether the driver steps on a brake of the vehicle, and sensing whether the driver grips a steering wheel of the vehicle.

The determining of whether the sobriety test of the driver is possible may include determining that the sobriety test of the driver is impossible when one or more of the actions of the driver are not sensed.

The method may further include re-sensing the actions of the driver and re-determining whether the sobriety test of the driver is possible based on the re-sensed actions.

The method may further include re-determining whether the sobriety test of the driver is possible when the driver is intoxicated.

The sensing of whether the driver fastens the seat belt in the driver's seat of the vehicle may include sensing whether a length of the seat belt in the driver's seat of the vehicle is changed by a predetermined length or more.

The method may further include, when it is sensed that the driver does not sit in the driver's seat of the vehicle while driving the vehicle, stopping the driving of the vehicle and re-determining whether the sobriety test of the driver is possible.

The method may further include, when it is sensed that not all the doors of the vehicle are closed while driving the vehicle, stopping the driving of the vehicle and re-determining whether the sobriety test of the driver is possible.

The method may further include, when it is sensed that the driver does not fasten the seat belt in the driver's seat of the vehicle while driving the vehicle, stopping the driving of the vehicle and re-determining whether the sobriety test of the driver is possible.

According to another aspect, there is provided an electronic device for preventing drunk driving of a driver who rides in a vehicle, the electronic device including a processor and a memory storing instructions, in which the instructions, when executed by the processor, cause the electronic device to sense actions of the driver, determine whether a sobriety test of the driver is possible based on a sensed result, obtain information on whether the driver is intoxicated when it is determined that the sobriety test of the driver is possible, control the vehicle based on the information, sense whether the driver sits in a driver's seat of the vehicle, sense whether the driver closes all doors of the vehicle, sense whether the driver fastens a seat belt in the driver's seat of the vehicle, sense whether the driver steps on a brake of the vehicle, and sense whether the driver grips a steering wheel of the vehicle.

The instructions, when executed by the processor, may cause the electronic device to determine that the sobriety test of the driver is impossible when one or more of the actions of the driver are not sensed.

The instructions, when executed by the processor, may cause the electronic device to re-sense the actions of the driver and re-determine whether the sobriety test of the driver is possible based on the re-sensed actions.

The instructions, when executed by the processor, may cause the electronic device to re-determine whether the sobriety test of the driver is possible when the driver is intoxicated.

The instructions, when executed by the processor, may cause the electronic device to sense whether a length of the seat belt in the driver's seat of the vehicle is changed by a predetermined length or more.

The instructions, when executed by the processor, may cause the electronic device to, when it is sensed that the driver does not sit in the driver's seat of the vehicle while driving the vehicle, stop the driving of the vehicle and re-determine whether the sobriety test of the driver is possible.

The instructions, when executed by the processor, may cause the electronic device to, when it is sensed that not all the doors of the vehicle are closed while driving the vehicle, stop the driving of the vehicle and re-determine whether the sobriety test of the driver is possible.

The instructions, when executed by the processor, may cause the electronic device to, when it is sensed that the driver does not fasten the seat belt in the driver's seat of the vehicle while driving the vehicle, stop the driving of the vehicle and re-determine whether the sobriety test of the driver is possible.

Additional aspects of embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a diagram illustrating a system for preventing drunk driving of a driver, according to an embodiment;

FIG. 2 is a diagram illustrating a breathalyzer according to an embodiment;

FIG. 3 is a diagram illustrating a monitoring device according to an embodiment;

FIG. 4 is a diagram illustrating a sensing module according to an embodiment;

FIG. 5 is a flowchart illustrating a method of determining whether a sobriety test of a driver is possible, according to an embodiment;

FIG. 6 is a flowchart illustrating a method of controlling a vehicle according to the result of a sobriety test of a driver, according to an embodiment;

FIG. 7 is a flowchart illustrating a method of re-determining whether a sobriety test of a driver is possible while driving a vehicle, according to an embodiment;

FIG. 8 is a flowchart illustrating a method of re-determining whether a sobriety test of a driver is possible while driving a vehicle, according to another embodiment;

FIG. 9 is a flowchart illustrating a method of re-determining whether a sobriety test of a driver is possible while driving a vehicle, according to another embodiment;

FIG. 10 is a flowchart illustrating a method of preventing drunk driving of a driver who rides in a vehicle, according to an embodiment; and

FIG. 11 is a schematic block diagram illustrating an electronic device according to an embodiment.

### DETAILED DESCRIPTION

The following detailed structural or functional description is provided as an example only and various alterations and modifications may be made to the embodiments. Accordingly, the embodiments are not construed as limited to the disclosure and should be understood to include all changes, equivalents, and replacements within the idea and the technical scope of the disclosure.

Although terms, such as first, second, and the like are used to describe various components, the components are not limited to the terms. These terms should be used only to distinguish one component from another component. For example, a first component may be referred to as a second component, and similarly the second component may also be referred to as the first component.

It should be noted that if one component is described as being “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. As used herein, “A or B”, “at least one of A and B”, “at least one of A or B”, “A, B or C”, “at least one of A, B and C”, and “at least one of A, B, or C,” each of which may include any one of the items listed together in the corresponding one of the phrases, or all possible combinations thereof. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which the present disclosure pertains. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

As used in connection with the present disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an example, the module may be implemented in a form of an application-specific integrated circuit (ASIC).

The term “unit” used herein may refer to a software or hardware component, such as a field-programmable gate array (FPGA) or an ASIC, and the “unit” performs predefined functions. However, “unit” is not limited to software or hardware. The “unit” may be configured to reside on an addressable storage medium or configured to operate one or more processors. Accordingly, the “unit” may include, for example, components, such as software components, object-oriented software components, class components, and task components, processes, functions, attributes, procedures, sub-routines, segments of program code, drivers, firmware, microcode, circuitry, data, databases, data structures, tables, arrays, and variables. The functionalities provided in the components and “units” may be combined into fewer components and “units” or may be further separated into additional components and “units.” Furthermore, the components and “units” may be implemented to operate on one or more central processing units (CPUs) within a device or a security multimedia card. In addition, “unit” may include one or more processors.

Hereinafter, the embodiments will be described in detail with reference to the accompanying drawings. When describing the embodiments with reference to the accompanying drawings, like reference numerals refer to like components, and any repeated description related thereto will be omitted.

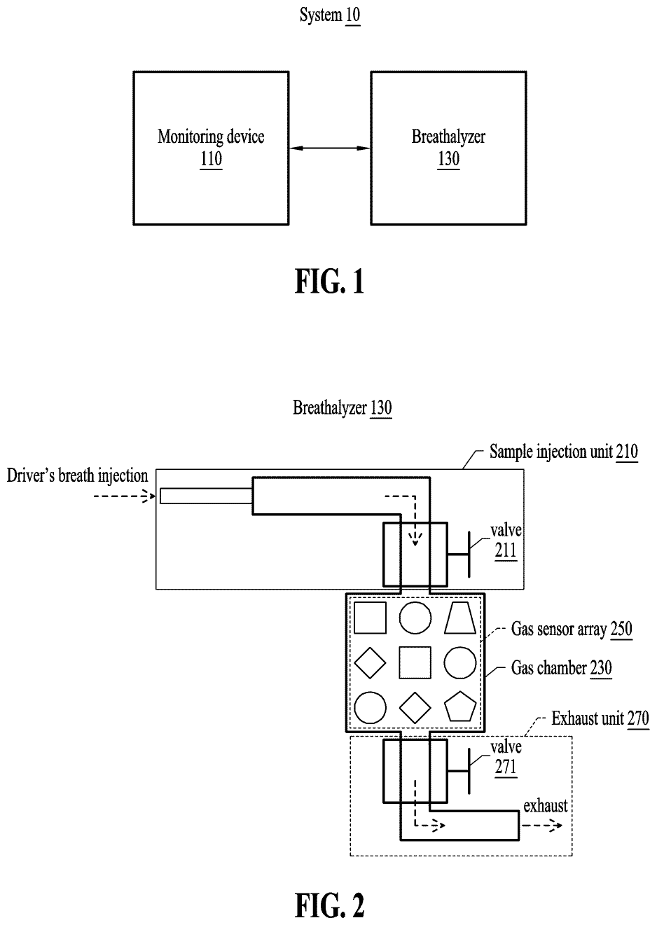

FIG. 1 is a diagram illustrating a system for preventing drunk driving of a driver, according to an embodiment.

Referring to FIG. 1, according to an embodiment, a system 10 may include a monitoring device 110 and a breathalyzer 130.

The monitoring device 110 may be used to monitor the actions of a driver who rides in a vehicle. The monitoring device 110 may be used to determine whether a sobriety test of the driver is possible by monitoring the actions of the driver. The monitoring device 110 may be an embedded vehicle device. For example, the monitoring device 110 may be an embedded vehicle smart device. The monitoring device 110 may be for the driver who rides in the vehicle to self-diagnose their driving ability. Based on the self-diagnosis result, the monitoring device 110 may warn the driver who rides in the vehicle of the risk of drunk driving. The monitoring device 110 may obtain information on whether the driver who rides in the vehicle is intoxicated from the breathalyzer 130. The monitoring device 110 may control the vehicle in which the driver is riding based on the obtained information. For example, when the information that the driver is intoxicated is obtained, the monitoring device 110 may control the vehicle so that the driving of the vehicle is impossible.

The breathalyzer 130 may be an apparatus for measuring whether the driver who rides in the vehicle is intoxicated. The breathalyzer 130 may obtain exhaled breath from the driver who rides in the vehicle. The breathalyzer 130 may detect an alcohol component from the obtained exhaled breath. The breathalyzer 130 may measure whether the driver who rides in the vehicle is intoxicated based on the detected result.

sample injection unit The currently implemented sobriety test method may be a method of measuring whether a driver is intoxicated by stopping all vehicles passing through a specific section. This method may have the disadvantage of mobilizing a large number of people on the road for a sobriety test. Accordingly, there may be a disadvantage that additional traffic accidents may occur due to the sobriety test. This sobriety test method may instill in the driver the perception that the driver is not caught in the crackdown on drunk driving as long as the driver avoids a specific section and time zone in which the sobriety test is performed. Accordingly, the effect of the crackdown on drunk driving may be reduced and drunk driving may not be prevented. In addition, this method may be interpreted as presuming all drivers on the road as potential drunk driving suspects, so there may be a possibility of human rights violations. To improve this sobriety test method, technology to prevent drunk driving using an embedded vehicle breathalyzer may be needed. Technology to prevent drunk driving using an embedded vehicle breathalyzer may control a vehicle so that the vehicle is not started when it is determined that a driver who rides in the vehicle is intoxicated through the breathalyzer. This method may not prevent a passenger from performing the sobriety test on behalf of the driver. Technology to prevent the driver in the driver's seat of the vehicle from drunk driving may be needed.

FIG. 2 is a diagram illustrating a breathalyzer according to an embodiment.

Referring to FIG. 2, according to an embodiment, the breathalyzer 130 may include a sample injection unit 210, a gas chamber 230, a gas sensor array 250, gas sensor circuitry (not shown), a display (not shown), an exhaust unit 270, and a mounting bracket (not shown).

The sample injection unit 210 may be a portion with which the oral cavity of the driver who rides in the vehicle is in contact. The sample injection unit 210 may be a portion into which the exhaled breath of the driver who rides in the vehicle is injected. For example, the sample injection unit 210 may have a shape such as a straw so that the exhaled breath of the driver may be injected. Accordingly, the sample injection unit 210 may have a shape into which the exhaled breath of the driver is smoothly injected at the predetermined pressure or more. The sample injection unit 210 may include a gas valve 211 and a gas pipe.

The gas chamber 230 may be a portion in which the injected exhaled breath of the driver is filled. Valves (e.g., a valve 211 and a valve 271) may be positioned at both ends of the gas chamber 230. The valves at both ends of the gas chamber 230 may be the gas valve 211 of the sample injection unit 210 and the gas valve 271 of the exhaust unit 270, respectively. When the exhaled breath of the driver is injected in a predetermined amount or more, the valves (e.g., the valve 211 and the valve 271) at both ends of the gas chamber 230 may be locked. Accordingly, the breathalyzer 130 may stably measure whether the driver is intoxicated.

The gas sensor array 250 may obtain the exhaled breath of the driver through the gas chamber 230. The gas sensor array 250 may include a plurality of gas sensors. For example, the gas sensor array 250 may be a gas sensor in a multi-modal type. The plurality of gas sensors may have a variety of shapes and/or methods. For example, the plurality of gas sensors may include a semiconductor sensor and/or an electrochemical sensor. Accordingly, the accuracy and sensitivity of alcohol component detection of the gas sensor array 250 may be improved. The gas sensor array 250 may detect alcohol from the exhaled breath of the driver. The gas sensor array 250 may measure whether the driver is intoxicated based on the detected result. The gas sensor array 250 may output a signal including the information on whether the driver is intoxicated based on the measured result.

The gas sensor circuitry may process an output signal of the gas sensor array 250. The gas sensor circuitry may transmit the information on whether the driver is intoxicated to the monitoring device 110 based on the output signal of the gas sensor array 250.

The display may display the measured result regarding whether the driver is intoxicated based on the output signal of the gas sensor array 250. For example, when it is measured that the driver is intoxicated, the display may guide the driver who rides in the vehicle to take the sobriety test again.

The exhaust unit 270 may be a portion through which the exhaled breath of the driver is exhausted. The breathalyzer 130 may exhaust the exhaled breath of the driver, which is filled in the gas chamber 230, through the exhaust unit 270. The exhaust unit 270 may include the gas valve 271 and the gas pipe.

The breathalyzer 130 may include a portable breathalyzer and an embedded vehicle breathalyzer. The breathalyzer 130 may measure alcohol from the exhaled breath of the driver using a gas sensor in a multi-modal type. Accordingly, the breathalyzer 130 may have higher accuracy and sensitivity in alcohol measurement than the existing breathalyzer that only uses an electrochemical gas sensor.

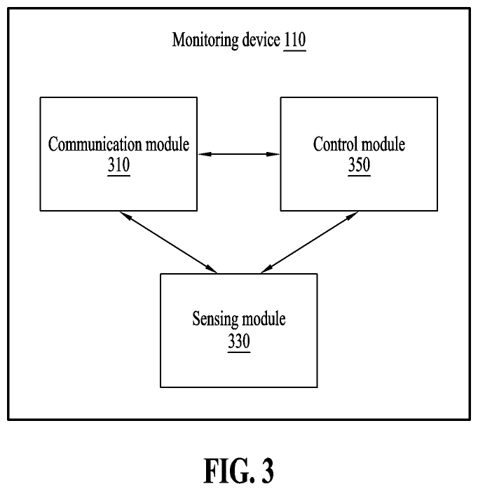

FIG. 3 is a diagram illustrating a monitoring device according to an embodiment.

Referring to FIG. 3, according to an embodiment, the monitoring device 110 may include a communication module 310, a sensing module 330, and a control module 350. The monitoring device 110 may sense whether a driver sits in the driver's seat of a vehicle, whether the driver closes all doors of the vehicle, whether the driver fastens a seat belt in the driver's seat of the vehicle, whether the driver steps on a brake of the vehicle, and whether the driver grips a steering wheel of the vehicle. The monitoring device 110 may determine that a sobriety test of the driver is impossible when one or more of the actions of the driver who rides in the vehicle are not sensed. The monitoring device 110 may sense the actions of the driver again when it is determined that the sobriety test of the driver is impossible. The monitoring device 110 may re-determine whether the sobriety test of the driver is possible based on the sensed actions.

The communication module 310 may be used to communicate between a mobile device owned by the driver and the vehicle. The communication module 310 may recognize that the mobile device owned by the driver approaches the vehicle. The communication module 310 may start the vehicle when the mobile device approaches the vehicle by a predetermined distance or less. The communication module 310 may transmit, to the breathalyzer 130, the sensing module 330, and the control module 350, a signal for preparing for the driving of the vehicle when the vehicle is started. The communication module 310, in particular cases, may stop the operation of the breathalyzer 130, the sensing module 330, and the control module 350 and may turn off the vehicle. The particular cases may include a case in which, after the vehicle is started, a predetermined time elapses without the driver riding the vehicle or the mobile device owned by the driver moves away from the vehicle by a predetermined distance or more.

The sensing module 330 may sense the actions of the driver who rides in the vehicle. Specifically, the sensing module 330 may sense an action that the driver who rides in the vehicle must perform during the sobriety test process. The sensing module 330 may include a seating sensing portion, a door sensing portion, a seat belt sensing portion, a brake sensing portion, a steering wheel sensing portion, and an image recognizer. The sensing module 330 may transmit a signal to the control module 350 based on the sensed actions of the driver.

The control module 350 may be used to control the breathalyzer 130 and the vehicle. The control module 350 may control the breathalyzer 130 and the vehicle based on the signal obtained from the sensing module 330. The control module 350 may control the vehicle so that the doors of the vehicle are opened and closed even when the driver does not perform the sobriety test. The control module 350 may control the vehicle so that the vehicle is started or turned off even when the driver does not perform the sobriety test. The control module 350 may control the vehicle not to move even when the driver steps on the accelerator when the driver does not perform the sobriety test. The control module 350 may control the breathalyzer 130 so that the operation of the breathalyzer 130 is stopped when a signal requesting control of the vehicle is obtained from the sensing module 330. The control module 350 may obtain information on whether the driver is intoxicated from the breathalyzer 130. When the driver is intoxicated, the control module 350 may control the operation of the breathalyzer 130 to stop. In this case, the control module 350 may control the breathalyzer 130 to make the sobriety test impossible for 10 minutes. When the information obtained from the breathalyzer 130 indicates that the driver is intoxicated three times in a row, the control module 350 may control the breathalyzer 130 so that the sobriety test is impossible for at least 12 hours. The control module 350 may control the vehicle not to move when, after the sobriety test is completed, the driver gets out of the driver's seat, the doors of the vehicle are opened, the buckle of the seat belt is released, or the length of the seat belt changes abnormally (e.g., more than a predetermined length). In such a case, the control module 350 may control the breathalyzer 130 and the vehicle so that the driver who rides in the vehicle performs the sobriety test again.

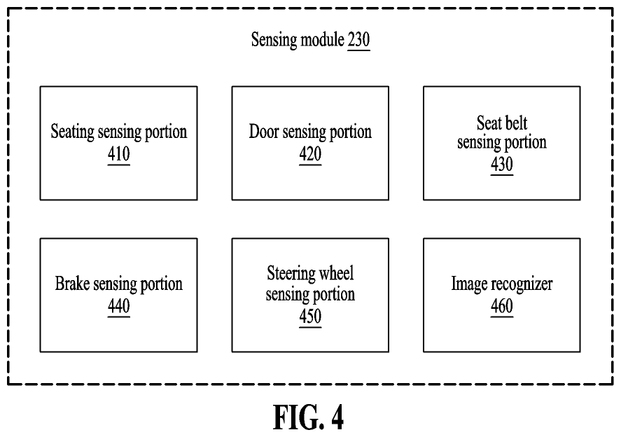

FIG. 4 is a diagram illustrating a sensing module according to an embodiment.

Referring to FIG. 4, according to an embodiment, the sensing module 330 may include a seating sensing portion 410, a door sensing portion 420, a seat belt sensing portion 430, a brake sensing portion 440, a steering wheel sensing portion 450, and an image recognizer 460.

The seating sensing portion 410 may be used to sense whether a driver sits in the driver's seat of a vehicle. The seating sensing portion 410 may include a pressure sensor and a motion sensor. The pressure sensor may be in the seat cushion of the driver's seat. The motion sensor may be in the backrest of the driver's seat. The seating sensing portion 410 may transmit a signal to the breathalyzer 130 so that the sobriety test is possible only when the driver sits in the driver's seat of the vehicle. The seating sensing portion 410 may transmit a signal to the control module 350 so that the sobriety test is stopped when the sobriety test of the driver starts and the pressure sensor does not sense the pressure above a predetermined level for more than 3 seconds. The seating sensing portion 410 may transmit a signal to the control module 350 so that the sobriety test is stopped when the sobriety test of the driver starts and nothing is sensed by the motion sensor. When the sobriety test is stopped, the driver who rides in the vehicle may re-perform the sobriety test after one minute. When the driver gets out of the vehicle after the sobriety test is completed, the seating sensing portion 410 may control the vehicle not to move by transmitting a signal to the control module 350. In such a case, the driver must re-perform the sobriety test after getting into the vehicle, and then, the driver may drive the vehicle.

The door sensing portion 420 may be used to sense whether all doors of the vehicle are closed. The door sensing portion 420 may sense whether the doors of the vehicle are closed through a door sensor. The door sensing portion 420 may transmit a signal to the breathalyzer 130 so that the sobriety test is impossible when not all the doors of the vehicle are closed. When the sobriety test starts and the door of the vehicle is opened, the door sensing portion 420 may transmit a signal to the control module 350 so that the sobriety test is stopped. When the sobriety test is stopped, the driver who rides in the vehicle may re-perform the sobriety test after one minute. When the door of the vehicle is opened after the sobriety test is completed, the door sensing portion 420 may control the vehicle not to move by transmitting a signal to the control module 350. In such a case, the driver must re-perform the sobriety test, and then, the driver may drive the vehicle.

The seat belt sensing portion 430 may be used to sense whether the driver who rides in the vehicle fastens a seat belt in the driver's seat. The seat belt sensing portion 430 may sense whether the driver fastens the seat belt in the driver's seat by sensing whether the buckle of the seat belt is fastened. In addition, the seat belt sensing portion 430 may sense whether the driver fastens the seat belt in the driver's seat by sensing whether the length of the seat belt in the driver's seat is changed by a predetermined length or more. For example, the seat belt sensing portion 430 may sense whether the driver fastens the seat belt in the driver's seat based on the length of the seat belt that is pulled when the driver fastens the seat belt. The seat belt sensing portion 430 may transmit a signal to the breathalyzer 130 so that the sobriety test is possible only when the driver who rides in the vehicle properly fastens the seat belt in the driver's seat. When the sobriety test starts and the buckle of the seat belt is released, the seat belt sensing portion 430 may transmit a signal to the control module 350 so that the sobriety test is stopped. When the sobriety test starts and the length of the seat belt is changed by a predetermined length or more, the seat belt sensing portion 430 may transmit a signal to the control module 350 so that the sobriety test is stopped. When the sobriety test is stopped, the driver who rides in the vehicle may re-perform the sobriety test after one minute. When the buckle of the seat belt is released after the sobriety test is completed, the seat belt sensing portion 430 may control the vehicle not to move by transmitting a signal to the control module 350. When the length of the seat belt is changed by a predetermined length or more after the sobriety test is completed, the seat belt sensing portion 430 may control the vehicle not to move by transmitting a signal to the control module 350. In such a case, the driver must re-perform the sobriety test, and then, the driver may drive the vehicle.

The brake sensing portion 440 may be used to sense whether the driver who rides in the vehicle steps on a brake of the vehicle. The brake sensing portion 440 may sense whether the driver steps on the brake of the vehicle through a brake sensor. The brake sensing portion 440 may transmit a signal to the breathalyzer 130 so that the sobriety test is possible only when the driver is stepping on the brake. When the sobriety test starts and the driver takes their foot off the brake, the brake sensing portion 440 may generate a warning signal (or warning sound). When the driver does not step on the brake while the warning signal is generated three times, the brake sensing portion 440 may transmit a signal to the control module 350 so that the sobriety test is stopped.

The steering wheel sensing portion 450 may be used to sense whether the driver who rides in the vehicle grips a steering wheel of the vehicle. The steering wheel sensing portion 450 may sense whether the driver grips the steering wheel of the vehicle through a grip sensor. The steering wheel sensing portion 450 may transmit a signal to the breathalyzer 130 so that the sobriety test is possible only when the driver grips the steering wheel. When the sobriety test starts and the driver does not grip the steering wheel, the steering wheel sensing portion 450 may generate a warning signal (or warning sound). When the driver does not grip the steering wheel while the warning signal is generated three times, the steering wheel sensing portion 450 may transmit a signal to the control module 350 so that the sobriety test is stopped.

The image recognizer 460 may be used to recognize the actions of the driver who rides in the vehicle. The image recognizer 460 may include a separate camera (e.g., a black box) installed inside the vehicle. The image recognizer 460 may capture an image of whether the driver who rides in the vehicle sits in the driver's seat through the camera. The image recognizer 460 may capture an image of whether all doors of the vehicle are closed through the camera. The image recognizer 460 may capture an image of whether the driver fastens a seat belt in the driver's seat through the camera. The image recognizer 460 may capture the actions of the driver during the sobriety test process and transmit a signal to the control module 350. The image recognizer 460 may capture and store the actions of the driver during the sobriety test process. The driver may not arbitrarily change or delete the stored image.

The sensing module 330 may sense the actions of the driver who rides in the vehicle and transmit a signal to the control module 350. The sensing module 330 may determine whether the sobriety test of the driver is possible based on the sensed result. The sensing module 330 may prevent a situation in advance in which a passenger performs the sobriety test on behalf of the driver by determining whether the sobriety test of the driver is possible. The sensing module 330 may prevent drunk driving in advance by allowing the driver in the driver's seat of the vehicle to directly perform the sobriety test.

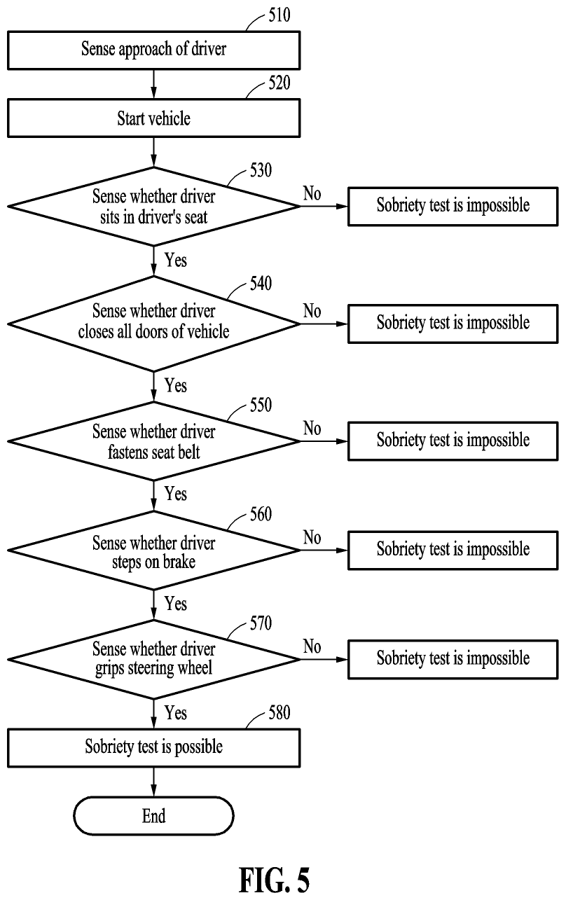

FIG. 5 is a flowchart illustrating a method of determining whether a sobriety test of a driver is possible, according to an embodiment.

Referring to FIG. 5, according to an embodiment, operations 510 to 580 may be performed by the monitoring device 110 (e.g., the monitoring device 110 of FIG. 1) described above with reference to FIGS. 1 to 4.

In operation 510, the monitoring device 110 may sense that the driver approaches the vehicle through the communication module 310.

In operation 520, the monitoring device 110 may start the vehicle when the driver approaches the vehicle by a predetermined distance or more.

In operation 530, the monitoring device 110 may sense whether the driver sits in the driver's seat of the vehicle through the sensing module 330. The monitoring device 110 may transmit a signal to the control module 350 so that the sobriety test is impossible when the driver does not sit in the driver's seat of the vehicle.

In operation 540, the monitoring device 110 may sense whether the driver closes all doors of the vehicle through the sensing module 330. The monitoring device 110 may transmit a signal to the control module 350 so that the sobriety test is impossible when the driver does not close the doors of the vehicle or when not all the doors of the vehicle are closed.

In operation 550, the monitoring device 110 may sense whether the driver fastens a seat belt in the driver's seat through the sensing module 330. The monitoring device 110 may transmit a signal to the control module 350 so that the sobriety test is impossible when the driver does not fasten the seat belt in the driver's seat.

In operation 560, the monitoring device 110 may sense whether the driver steps on a brake of the vehicle through the sensing module 330. The monitoring device 110 may transmit a signal to the control module 350 so that the sobriety test is impossible when the driver does not step on the brake of the vehicle.

In operation 570, the monitoring device 110 may sense whether the driver grips a steering wheel of the vehicle through the sensing module 330. The monitoring device 110 may transmit a signal to the control module 350 so that the sobriety test is impossible when the driver does not grip the steering wheel of the vehicle.

In operation 580, when operations 510 to 570 are all sensed, the monitoring device 110 may transmit a signal to the control module 350 so that the sobriety test of the driver who rides in the vehicle is possible.

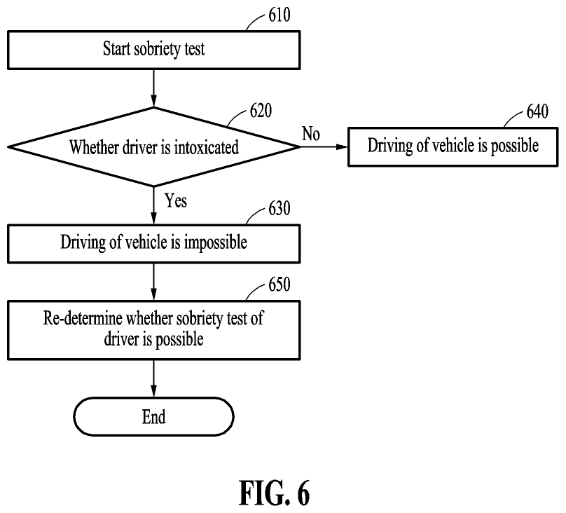

FIG. 6 is a flowchart illustrating a method of controlling a vehicle according to the result of a sobriety test of a driver, according to an embodiment.

Referring to FIG. 6, according to an embodiment, operations 610 to 650 may be substantially the same as a method of controlling a vehicle based on information on whether a driver is intoxicated.

In operation 610, the monitoring device 110 may transmit a signal to the breathalyzer 130 through the control module 350 so that the sobriety test of the driver who rides in the vehicle is possible. The breathalyzer 130 may start the sobriety test of the driver.

In operation 620, the monitoring device 110 may obtain the information on whether the driver is intoxicated from the breathalyzer 130.

In operation 630, when the driver is intoxicated, the monitoring device 110 may control the vehicle through the control module 350 so that the driving of the vehicle is impossible.

In operation 640, when the driver is not intoxicated, the monitoring device 110 may control the vehicle through the control module 350 so that the driving of the vehicle is possible.

In operation 650, the monitoring device 110 may re-determine whether the sobriety test of the driver is possible. The monitoring device 110 may transmit a signal to the control module 350 so that the sobriety test of the driver who rides in the vehicle is possible when operations 510 to 570 (operations 510 to 570 of FIG. 5) are all sensed.

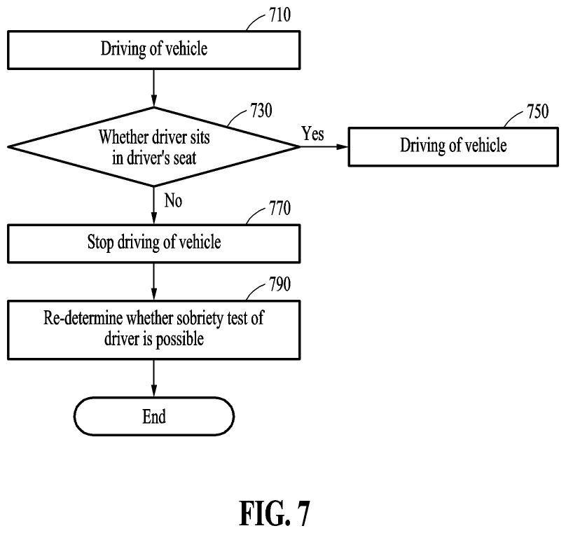

FIG. 7 is a flowchart illustrating a method of re-determining whether a sobriety test of a driver is possible while driving a vehicle, according to an embodiment.

In operation 710, when the driver who rides in the vehicle is not intoxicated, the monitoring device 110 may control the vehicle through the control module 350 so that the driving of the vehicle is possible.

In operation 730, the monitoring device 110 may sense whether the driver sits in the driver's seat through the sensing module 330 while driving the vehicle.

In operation 750, when the monitoring device 110 senses that the driver sits in the driver's seat, the monitoring device 110 may control the vehicle through the control module 350 so that the driving of the vehicle continues.

In operation 770, when the monitoring device 110 senses that the driver does not sit in the driver's seat, the monitoring device 110 may control the vehicle through the control module 350 so that the driving of the vehicle is stopped.

In operation 790, the monitoring device 110 may re-determine whether the sobriety test of the driver is possible. The monitoring device 110 may transmit a signal to the control module 350 so that the sobriety test of the driver who rides in the vehicle is possible when operations 510 to 570 (operations 510 to 570 of FIG. 5) are all sensed.

The monitoring device 110 may re-determine whether the sobriety test of the driver in the driver's seat is possible when a change in state of the vehicle occurs while driving the vehicle (e.g., the driver gets out of the vehicle). The monitoring device 110 may re-determine whether the sobriety test of the driver is possible, thereby preventing a passenger or the like, not the driver, from performing the sobriety test on behalf of the driver.

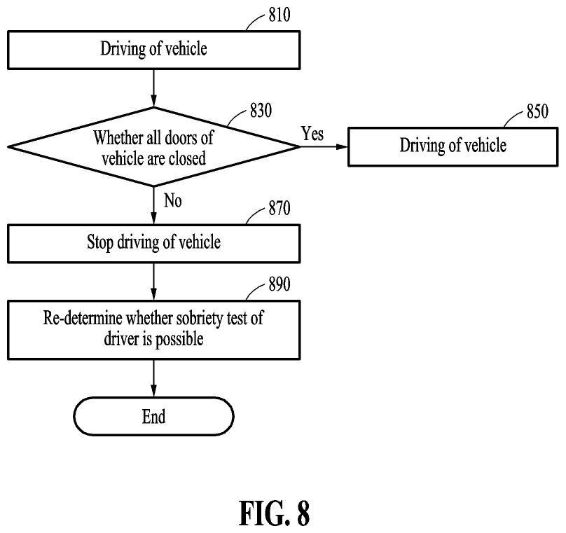

FIG. 8 is a flowchart illustrating a method of re-determining whether a sobriety test of a driver is possible while driving a vehicle, according to another embodiment.

In operation 810, when the driver who rides in the vehicle is not intoxicated, the monitoring device 110 may control the vehicle through the control module 350 so that the driving of the vehicle is possible.

In operation 830, the monitoring device 110 may sense whether all doors of the vehicle are closed through the sensing module 330 while driving the vehicle.

In operation 850, when the monitoring device 110 senses that all doors of the vehicle are closed, the monitoring device 110 may control the vehicle through the control module 350 so that the driving of the vehicle continues.

In operation 870, when the monitoring device 110 senses that not all the doors of the vehicle are closed, the monitoring device 110 may control the vehicle through the control module 350 so that the driving of the vehicle is stopped.

In operation 890, the monitoring device 110 may re-determine whether the sobriety test of the driver is possible. The monitoring device 110 may transmit a signal to the control module 350 so that the sobriety test of the driver who rides in the vehicle is possible when operations 510 to 570 (operations 510 to 570 of FIG. 5) are all sensed.

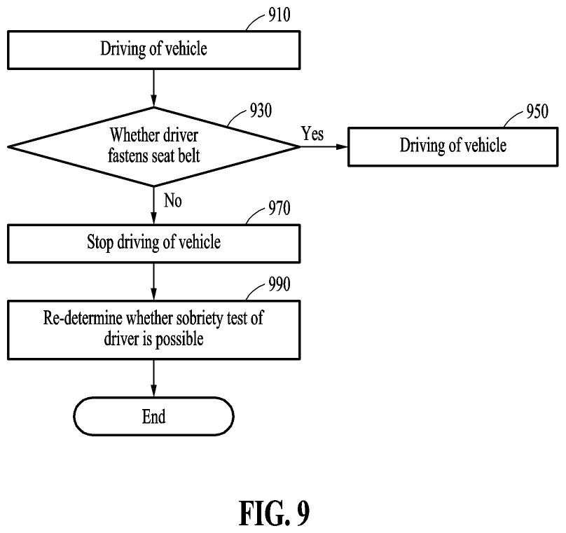

FIG. 9 is a flowchart illustrating a method of re-determining whether a sobriety test of a driver is possible while driving a vehicle, according to another embodiment.

In operation 910, when the driver who rides in the vehicle is not intoxicated, the monitoring device 110 may control the vehicle through the control module 350 so that the driving of the vehicle is possible.

In operation 930, the monitoring device 110 may sense whether the driver fastens a seat belt in the driver's seat through the sensing module 330 while driving the vehicle.

In operation 950, when the monitoring device 110 senses that the driver fastens the seat belt in the driver's seat, the monitoring device 110 may control the vehicle through the control module 350 so that the driving of the vehicle continues.

In operation 970, when the monitoring device 110 senses that the driver does not fasten the seat belt in the driver's seat, the monitoring device 110 may control the vehicle through the control module 350 so that the driving of the vehicle is stopped.

In operation 990, the monitoring device 110 may re-determine whether the sobriety test of the driver is possible. The monitoring device 110 may transmit a signal to the control module 350 so that the sobriety test of the driver who rides in the vehicle is possible when operations 510 to 570 (operations 510 to 570 of FIG. 5) are all sensed.

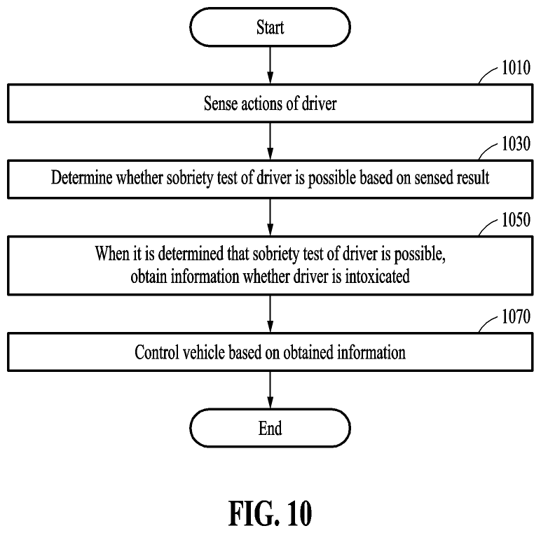

FIG. 10 is a flowchart illustrating a method of preventing drunk driving of a driver who rides in a vehicle, according to an embodiment.

Referring to FIG. 10, according to an embodiment, the monitoring device 110 described above with reference to FIGS. 1 to 9 may monitor the actions of the driver who rides in the vehicle.

In operation 1010, the monitoring device 110 may sense the actions of the driver who rides in the vehicle.

In operation 1030, the monitoring device 110 may determine whether a sobriety test of the driver is possible based on the sensed result.

In operation 1050, when it is determined that the sobriety test of the driver is possible, the monitoring device 110 may obtain information on whether the driver is intoxicated.

In operation 1070, the monitoring device 110 may control the vehicle based on the obtained information.

Operations 1010 to 1070 may be performed sequentially, but embodiments are not limited thereto. For example, two or more operations may be performed in parallel. Operations 1010 to 1070 may be substantially the same as the method of preventing drunk driving of a driver who rides in a vehicle, according to an embodiment, which is performed by the monitoring device 110 described above with reference to FIGS. 1 to 9.

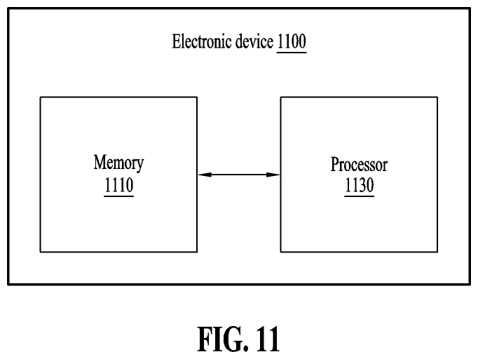

FIG. 11 is a schematic block diagram illustrating an electronic device according to an embodiment.

Referring to FIG. 11, according to an embodiment, an electronic device 1100 may include a memory 1110 and a processor 1130.

The memory 1110 may store instructions (or programs) executable by the processor 1130. For example, the instructions may include instructions for performing an operation of the processor 1130 and/or an operation of each component of the processor 1130.

The memory 1110 may include one or more of computer-readable storage media. The memory 1110 may include non-volatile storage elements (e.g., a magnetic hard disc, an optical disc, a floppy disc, flash memory, electrically programmable memory (EPROM), and electrically erasable and programmable memory (EEPROM)).

The memory 1110 may be a non-transitory medium. The term “non-transitory” may indicate that a storage medium is not embodied in a carrier wave or a propagated signal. However, the term “non-transitory” should not be interpreted to mean that the memory 1110 is non-movable.

The processor 1130 may process data stored in the memory 1110. The processor 1130 may execute computer-readable code (e.g., software) stored in the memory 1110 and instructions triggered by the processor 1130.

The processor 1130 may be a data-processing device implemented by hardware having a circuit that has a physical structure to execute desired operations. For example, the desired operations may include code or instructions included in a program.

For example, the data-processing device implemented by hardware may include a microprocessor, a CPU, a processor core, a multi-core processor, a multiprocessor, an ASIC, and an FPGA.

The processor 1130 may cause the electronic device 1100 to perform one or more operations by executing the code and/or instructions stored in the memory 1110. The operations performed by the electronic device 1100 may be substantially the same as the operations performed by the monitoring device 110 described above with reference to FIGS. 1 to 10. Accordingly, a repeated description thereof is omitted.

The embodiments described herein may be implemented using a hardware component, a software component and/or a combination thereof. A processing device may be implemented using one or more general-purpose or special-purpose computers, such as, for example, a processor, a controller and an arithmetic logic unit (ALU), a digital signal processor (DSP), a microcomputer, an FPGA, a programmable logic unit (PLU), a microprocessor, or any other device capable of responding to and executing instructions in a defined manner. The processing device may run an operating system (OS) and one or more software applications that run on the OS. The processing device also may access, store, manipulate, process, and generate data in response to execution of the software. For purpose of simplicity, the description of a processing device is singular; however, one of ordinary skill in the art will appreciate that a processing device may include a plurality of processing elements and a plurality of types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or collectively instruct or configure the processing device to operate as desired. Software and data may be stored in any type of machine, component, physical or virtual equipment, or computer storage medium or device capable of providing instructions or data to or being interpreted by the processing device. The software may also be distributed over network-coupled computer systems so that the software is stored and executed in a distributed fashion. The software and data may be stored in a non-transitory computer-readable recording medium.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as optical discs; and hardware devices that are specifically configured to store and perform program instructions, such as read-only memory (ROM), random access memory (RAM), flash memory, and the like. Examples of program instructions include both machine code, such as one produced by a compiler, and files containing higher-level code that may be executed by the computer using an interpreter.

The above-described hardware devices may be configured to act as one or more software modules in order to perform the operations of the above-described embodiments, or vice versa.

As described above, although the embodiments have been described with reference to the limited drawings, one of ordinary skill in the art may apply various technical modifications and variations based thereon. For example, suitable results may be achieved if the described techniques are performed in a different order, and/or if components in a described system, architecture, device, or circuit are combined in a different manner, and/or replaced or supplemented by other components or their equivalents.

Therefore, other implementations, other embodiments, and equivalents to the claims are also within the scope of the following claims.

What is claimed is:

1. A method of preventing drunk driving of a driver who rides in a vehicle, the method comprising:
sensing actions of the driver;
determining whether a sobriety test of the driver is possible based on a sensed result;
obtaining information on whether the driver is intoxicated when it is determined that the sobriety test of the driver is possible; and
controlling the vehicle based on the information,
wherein the sensing of the actions comprises:
sensing whether the driver sits in a driver's seat of the vehicle;
sensing whether the driver closes all doors of the vehicle;
sensing whether the driver fastens a seat belt in the driver's seat of the vehicle;
sensing whether the driver steps on a brake of the vehicle; and
sensing whether the driver grips a steering wheel of the vehicle.

2. The method of claim 1, wherein the determining of whether the sobriety test of the driver is possible comprises determining that the sobriety test of the driver is impossible when one or more of the actions of the driver are not sensed.

3. The method of claim 2, further comprising:
re-sensing the actions of the driver; and
re-determining whether the sobriety test of the driver is possible based on the re-sensed actions.

4. The method of claim 1, further comprising:
re-determining whether the sobriety test of the driver is possible when the driver is intoxicated.

5. The method of claim 1, wherein the sensing of whether the driver fastens the seat belt in the driver's seat of the vehicle comprises sensing whether a length of the seat belt in the driver's seat of the vehicle is changed by a predetermined length or more.

6. The method of claim 1, further comprising:
when it is sensed that the driver does not sit in the driver's seat of the vehicle while driving the vehicle,
stopping the driving of the vehicle; and
re-determining whether the sobriety test of the driver is possible.

7. The method of claim 1, further comprising:
when it is sensed that not all the doors of the vehicle are closed while driving the vehicle,
stopping the driving of the vehicle; and
re-determining whether the sobriety test of the driver is possible.

8. The method of claim 1, further comprising:
when it is sensed that the driver does not fasten the seat belt in the driver's seat of the vehicle while driving the vehicle,
stopping the driving of the vehicle; and
re-determining whether the sobriety test of the driver is possible.

9. The method of claim 1, wherein the obtaining of the information comprises:
detecting alcohol from exhaled breath from the driver; and
obtaining the information on whether the driver is intoxicated based on a detected result.

10. The method of claim 1, wherein the information on whether the driver is intoxicated is obtained based on an output signal of a gas sensor array comprising a plurality of gas sensors.

11. An electronic device for preventing drunk driving of a driver who rides in a vehicle, the electronic device comprising:
a processor; and
a memory storing instructions,
wherein the instructions, when executed by the processor, cause the electronic device to:
sense actions of the driver;
determine whether a sobriety test of the driver is possible based on a sensed result;
obtain information on whether the driver is intoxicated when it is determined that the sobriety test of the driver is possible;
control the vehicle based on the information;
sense whether the driver sits in a driver's seat of the vehicle;
sense whether the driver closes all doors of the vehicle;
sense whether the driver fastens a seat belt in the driver's seat of the vehicle;
sense whether the driver steps on a brake of the vehicle; and
sense whether the driver grips a steering wheel of the vehicle.

12. The electronic device of claim 11, wherein the instructions, when executed by the processor, cause the electronic device to determine that the sobriety test of the driver is impossible when one or more of the actions of the driver are not sensed.

13. The electronic device of claim 12, wherein the instructions, when executed by the processor, cause the electronic device to re-sense the actions of the driver and re-determine whether the sobriety test of the driver is possible based on the re-sensed actions.

14. The electronic device of claim 11, wherein the instructions, when executed by the processor, cause the electronic device to re-determine whether the sobriety test of the driver is possible when the driver is intoxicated.

15. The electronic device of claim 11, wherein the instructions, when executed by the processor, cause the electronic device to sense whether a length of the seat belt in the driver's seat of the vehicle is changed by a predetermined length or more.

16. The electronic device of claim 11, wherein the instructions, when executed by the processor, cause the electronic device to, when it is sensed that the driver does not sit in the driver's seat of the vehicle while driving the vehicle:
stop the driving of the vehicle; and
re-determine whether the sobriety test of the driver is possible.

17. The electronic device of claim 11, wherein the instructions, when executed by the processor, cause the electronic device to, when it is sensed that not all the doors of the vehicle are closed while driving the vehicle:
stop the driving of the vehicle; and
re-determine whether the sobriety test of the driver is possible.

18. The electronic device of claim 11, wherein the instructions, when executed by the processor, cause the electronic device to, when it is sensed that the driver does not fasten the seat belt in the driver's seat of the vehicle while driving the vehicle:
stop the driving of the vehicle; and
re-determine whether the sobriety test of the driver is possible.

19. The electronic device of claim 11, wherein the instructions, when executed by the processor, cause the electronic device to:
detect alcohol from exhaled breath from the driver; and
obtain information on whether the driver is intoxicated based on a detected result.

20. The electronic device of claim 11, wherein the information on whether the driver is intoxicated is obtained based on an output signal of a gas sensor array comprising a plurality of gas sensors.
